(19) United States
(12) Patent Application Publication
(10) Pub. No.: US 2026/0047882 A1
(43) Pub. Date: Feb. 19, 2026

(54) JAW FEATURES OF MEDICAL INSTRUMENT END EFFECTOR

(71) Applicant: Cilag GmbH International, Zug (CH)

(72) Inventors: Tyler N. Brehm, Arcanum, OH (US); Jason R. Lesko, Cincinnati, OH (US); Adam E. Miller, Cincinnati, OH (US); Andrew S. Meyers, Cincinnati, OH (US)

(21) Appl. No.: 18/804,252

(22) Filed: Aug. 14, 2024

(51) Int. Cl. A61B 18/14; A61B 18/00

(52) U.S. Cl. CPC A61B 18/1445; A61B 2018/00601; A61B 2018/00619; A61B 2018/0063; A61B 2018/1452

(57) ABSTRACT

An apparatus includes a body, a shaft assembly, and an end effector. The shaft assembly includes an inner guide having a laterally facing surface and a distally facing surface. The end effector includes a first jaw and a second jaw. The first jaw is movable toward and away from the second jaw. The second jaw includes a first proximally projecting beam. The first proximally projecting beam is positioned and configured to engage the laterally facing surface of the inner guide to thereby establish and maintain a predetermined angular position of the end effector relative to the shaft assembly. The first proximally projecting beam is further positioned and configured to engage the distally facing surface of the inner guide to thereby establish and maintain a predetermined longitudinal position of the end effector relative to the shaft assembly.

### BACKGROUND

A variety of surgical instruments include a tissue cutting element and one or more elements that transmit radio frequency (RF) energy to tissue (e.g., to coagulate or seal the tissue). Examples of such electrosurgical instruments and related concepts are disclosed in U.S. Pat. No. 6,500,176 entitled “Electrosurgical Systems and Techniques for Sealing Tissue,” issued December 31, 2002, the disclosure of which is incorporated by reference herein, in its entirety; U.S. Pat. No. 9,526,565, entitled “Electrosurgical Devices,” issued December 27, 2016, the disclosure of which is incorporated by reference herein, in its entirety; U.S. Pat. No. 11,857,247, entitled “Jaw for Surgical Instrument End Effector,” issued January 2, 2024, the disclosure of which is incorporated by reference herein, in its entirety; and U.S. Pub. No. 2022/0008120, entitled “Electrosurgical Instrument with Floating Jaw Component,” published January 13, 2022, the disclosure of which is incorporated by reference herein in its entirety.

An electrosurgical instrument may be powered by an external generator. Examples of such generators are disclosed in U.S. Pat. No. 8,986,302, entitled “Surgical Generator for Ultrasonic and Electrosurgical Devices,” issued March 24, 2015, the disclosure of which is incorporated by reference herein, in its entirety.

Some electrosurgical instruments may be supported and driven by a robotic surgical system. Examples of robotically controlled electrosurgical instruments are disclosed in U.S. Pat. No. 11,576,738, entitled “Systems and Instruments for Tissue Sealing,” issued February 14, 2023, the disclosure of which is incorporated by reference herein in its entirety; and U.S. Pub. No. 2022/0338891, entitled “Systems for Setting Jaw Gap in Surgical Tool End Effectors,” published October 27, 2022, the disclosure of which is incorporated by reference herein in its entirety.

While some manufacturing variations may be acceptable within certain tolerances, it may be desirable to minimize such variations and tolerances, particularly in the context of electrosurgical instruments. For instance, it may be desirable to ensure that the fit between an end effector of an electrosurgical instrument and a shaft assembly of the electrosurgical instrument are as consistent and predictable as possible. Minimizing manufacturing tolerances may minimize undesirable, unintended relative movement between components of the instrument. Similarly, minimizing manufacturing tolerances may provide greater consistency and precision in the permitted range of motion of certain movable components of the instrument, such that the motion of such components may be consistently and predictably arrested at predetermined positions during use of the instrument. As part of an effort to minimize manufacturing variations, it may be desirable to ensure that two or more components are consistently and precisely positioned relative to each other before such components are fixed together.

While a variety of surgical instruments have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements.

FIG. 1 depicts a schematic view of an example of an electrosurgical instrument

FIG. 2A depicts a perspective view of an end effector and distal portion of a shaft assembly of the instrument of FIG. 1, with the end effector in a closed configuration.

FIG. 2B depicts a perspective view of the end effector and distal portion of the shaft assembly of FIG. 2A, with the end effector in an open configuration and a knife in a proximal position.

FIG. 2C depicts a perspective view of the end effector and distal portion of the shaft assembly of FIG. 2A, with the end effector in the open configuration and the knife in a distal position.

FIG. 3A depicts a side elevation view of the end effector and distal portion of the shaft assembly of FIG. 2A, with an outer shaft of the shaft assembly omitted, and with the end effector in the closed configuration.

FIG. 3B depicts a side elevation view of the end effector and distal portion of the shaft assembly of FIG. 2A, with the outer shaft of the shaft assembly omitted, and with the end effector in the open configuration.

FIG. 4 depicts a perspective view of a first jaw of the end effector of the instrument of FIG. 1.

FIG. 5 depicts a perspective view of a second jaw of the end effector of the instrument of FIG. 1.

FIG. 6 depicts an exploded perspective view of the shaft assembly of the instrument of FIG. 1.

FIG. 7 depicts an exploded perspective view of a distal portion of the shaft assembly of the instrument of FIG. 1.

FIG. 8A depicts a perspective view of the end effector and distal portion of the shaft assembly of FIG. 2A, with an outer tube of the shaft assembly omitted for clarity, and with the end effector distally separated from the shaft assembly during a first stage of manufacture.

FIG. 8B depicts a perspective view of the end effector and distal portion of the shaft assembly of FIG. 2A, with an outer tube of the shaft assembly omitted for clarity, and with the end effector coupled with the shaft assembly during a second stage of manufacture.

FIG. 9A depicts a perspective view of the end effector and distal portion of the shaft assembly of FIG. 2A, with an outer tube of the shaft assembly omitted for clarity, and with a knife assembly of the shaft assembly in a proximal position during a first stage of operation.

FIG. 9B depicts a perspective view of the end effector and distal portion of the shaft assembly of FIG. 2A, with an outer tube of the shaft assembly omitted for clarity, and with the knife assembly of the shaft assembly in a distal position during a second stage of operation.

FIG. 10 depicts a perspective view of the end effector of the instrument of FIG. 1.

FIG. 11 depicts another perspective view of the end effector of FIG. 10.

FIG. 12 depicts a perspective view of the end effector and distal portion of the shaft assembly of FIG. 2A.

FIG. 13 depicts another perspective view of the end effector and distal portion of the shaft assembly of FIG. 2A.

FIG. 14 depicts a cross-sectional view of the perspective view of the end effector and distal portion of the shaft assembly of FIG. 2A, taken along line 14-14 of FIG. 12.

FIG. 15 depicts a cross-sectional view of the perspective view of the end effector and distal portion of the shaft assembly of FIG. 2A, taken along line 15-15 of FIG. 12.

FIG. 16A depicts a side elevation view of the end effector of FIG. 10 engaging a fixture during a first sub-stage of an example of a manufacturing process of the instrument of FIG. 1.

FIG. 16B depicts a side elevation view of the shaft assembly of the instrument of FIG. 1 being urged into engagement with the end effector of FIG. 10, while the end effector remains engaged with the fixture of FIG. 16A, during a second sub-stage of an example of a manufacturing process of the instrument of FIG. 1.

FIG. 16C depicts a side elevation view of the shaft assembly of the instrument of FIG. 1 being secured to the end effector of FIG. 10, while the end effector remains engaged with the fixture of FIG. 16A, during a third sub-stage of an example of a manufacturing process of the instrument of FIG. 1.

FIG. 17A depicts a side elevation view of the shaft assembly of the instrument of FIG. 1 engaging a fixture during a first sub-stage of an example of a manufacturing process of the instrument of FIG. 1.

FIG. 17B depicts a side elevation view of the body of the instrument of FIG. 1 being secured to the shaft assembly, while the shaft assembly remains engaged with the fixture of FIG. 17A, during a second sub-stage of an example of a manufacturing process of the instrument of FIG. 1.

### DETAILED DESCRIPTION

Any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

### I. Overview of Electrosurgical Instrument

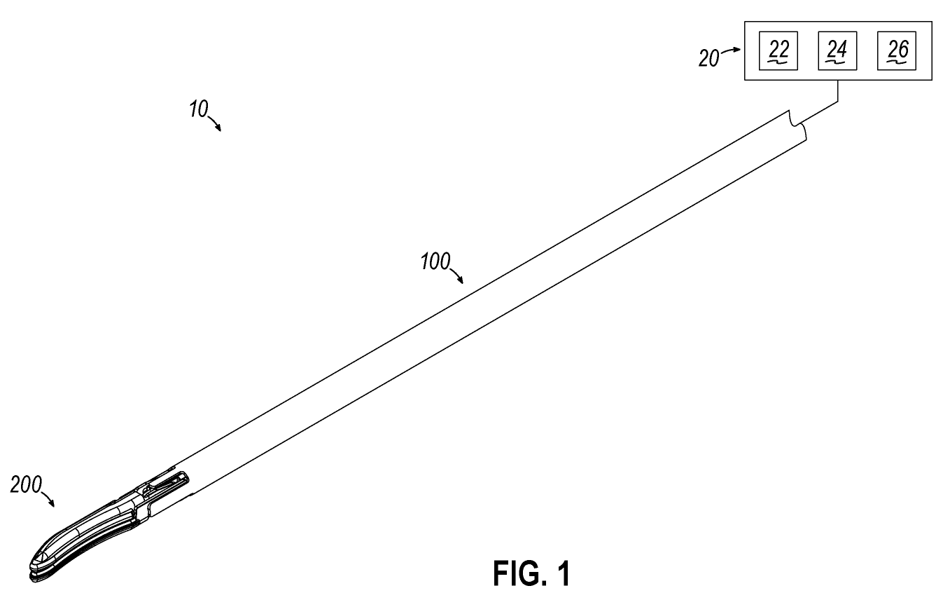

FIG. 1 shows an example of an electrosurgical instrument (10) that may be used in numerous kinds of medical procedures. Instrument (10) of this example includes a body assembly (20), a shaft assembly (100), and an end effector (200). Shaft assembly (100) extends distally relative to body assembly (20). End effector (200) is positioned at a distal end of shaft assembly (100). As will be described in greater detail below, end effector (200) is operable to grasp, cut, and seal or weld tissue (e.g., a blood vessel, etc.). Specifically, end effector (200) is operable to grasp tissue between opposing jaws (220, 240), cut the tissue with a knife (184), and seal or weld the tissue by applying bipolar RF energy to the tissue via respective electrodes (224, 244) of jaws (220, 240). While not shown in FIG. 1, instrument (10) may be coupled with a generator that is operable to provide the RF energy to electrodes (224, 244).

Body assembly (20) may take numerous different forms. In some versions, body assembly (20) includes a handle, such that body assembly (20) is configured to be grasped and manipulated by at least one hand of a human operator. By way of example only, such a handle may include a pistol grip, a scissor grip, or any other suitable configuration. In some other versions, body assembly (20) is configured to be coupled with a robotic arm or other component of a robotic surgical system, such that body assembly (20) is configured to be supported and activated by the robotic surgical system. Alternatively, body assembly (20) may take any other suitable form.

Body assembly (20) of the present example includes a jaw closure assembly (22), a knife drive assembly (24), and an input interface (26). Jaw closure assembly (22) is operable to drive jaws (220, 240) of end effector between an open position and a closed position, with closure motion from jaw closure assembly (22) being communicated to end effector (200) via shaft assembly (100) as will be described in greater detail below. Any suitable components and arrangements may be used to form jaw closure assembly (22) as will be apparent to those skilled in the art in view of the teachings herein. Knife drive assembly (24) is operable to drive knife (184) between a proximal position and a distal position, with translational motion from knife drive assembly (24) being communicated to end effector (200) via shaft assembly (100) as will be described in greater detail below. Any suitable components and arrangements may be used to form knife drive assembly (24) as will be apparent to those skilled in the art in view of the teachings herein.

Input interface (26) is operable to interface with a human or robotic operator to activate jaw closure assembly (22), knife drive assembly (24), and/or communication of RF energy to electrodes (224, 244). In versions where body assembly (30) is configured to be grasped and manipulated by at least one hand of a human operator, input interface (26) may include one or more trigger, one or more levers, one or more sliders, one or more buttons, and/or any other suitable kind(s) of input features, including combinations thereof. In versions where body assembly (30) is configured to be coupled with a robotic arm or other component of a robotic surgical system, input interface (26) may include one or more gears, one or more drive spindles, one or more shuttles or other translating drive elements, and/or other kind(s) of features that mechanically interface with complementary features of the robotic arm or other component of the robotic surgical system. In addition, or in the alternative, in versions where body assembly (30) is configured to be coupled with a robotic arm or other component of a robotic surgical system, input interface (26) may include one or more pins or sockets, one or more other kinds of electrical contacts, and/or any other kind(s) of features that electrically interface with complementary features of the robotic arm or other component of the robotic surgical system.

### A. Overview of End Effector

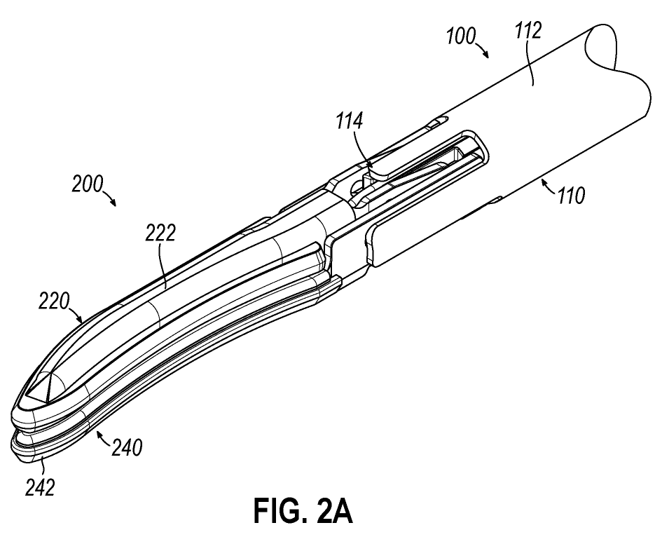

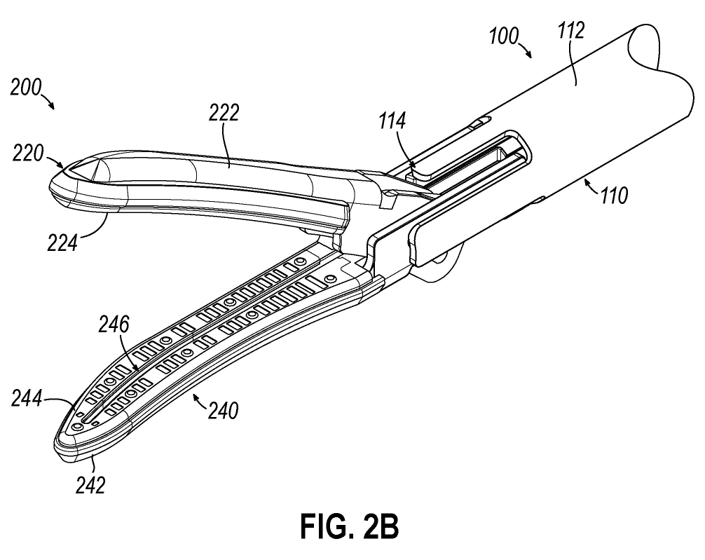

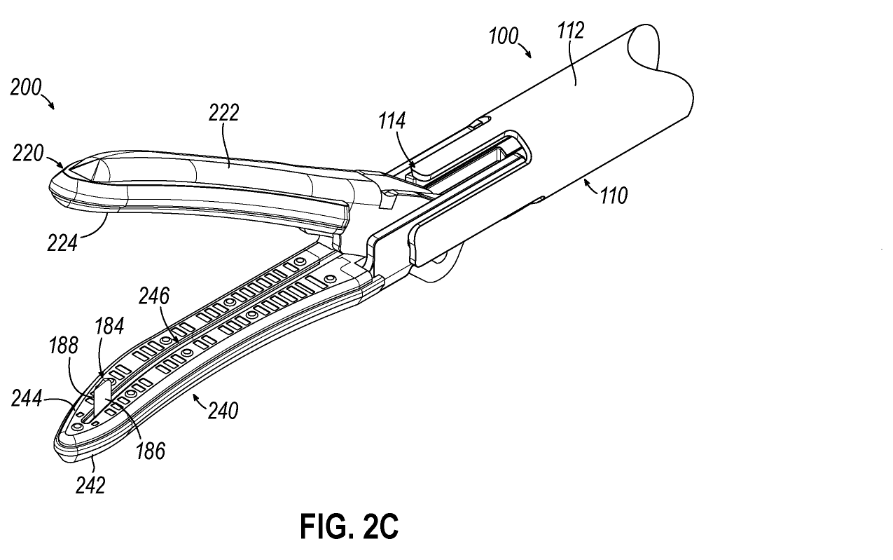

As shown in FIGS. 2A-2C, and as noted above, end effector (200) of the present example includes a first jaw (220) and a second jaw (240) that are operable to transition between a closed configuration (FIG. 2A) and an open configuration (FIG. 2B). In the present example, jaw (220) pivots toward and away from jaw (240), while jaw (240) remains stationary relative to shaft assembly (100), as jaws (220, 240) transition between the open configuration and the closed configuration. In some other versions, both jaws (220, 240) pivot simultaneously toward and away from each other in opposing fashion to transition between the open configuration and the closed configuration. Alternatively, one or both of jaws (220, 240) may move in any other suitable fashion.

As shown in the transition from FIG. 2B to FIG. 2C, knife (184) is operable to translate relative to jaws (220, 240) from a proximal position (FIG. 2B) to a distal position (FIG. 2C). While knife (184) is shown as being in the distal position while jaws (220, 240) are in the open configuration, some versions of operation may provide translation of knife (184) from the proximal position to the distal position only when jaws (220, 240) are in the closed configuration. In other words, in some versions of instrument (10) and methods of use of instrument (10), knife (184) would not be in the distal position when jaws (220, 240) are in the open configuration. The arrangement shown in FIG. 2C should therefore be understood as only being provided for purposes of illustration to show a distal position of knife (184) in relation to jaw (240).

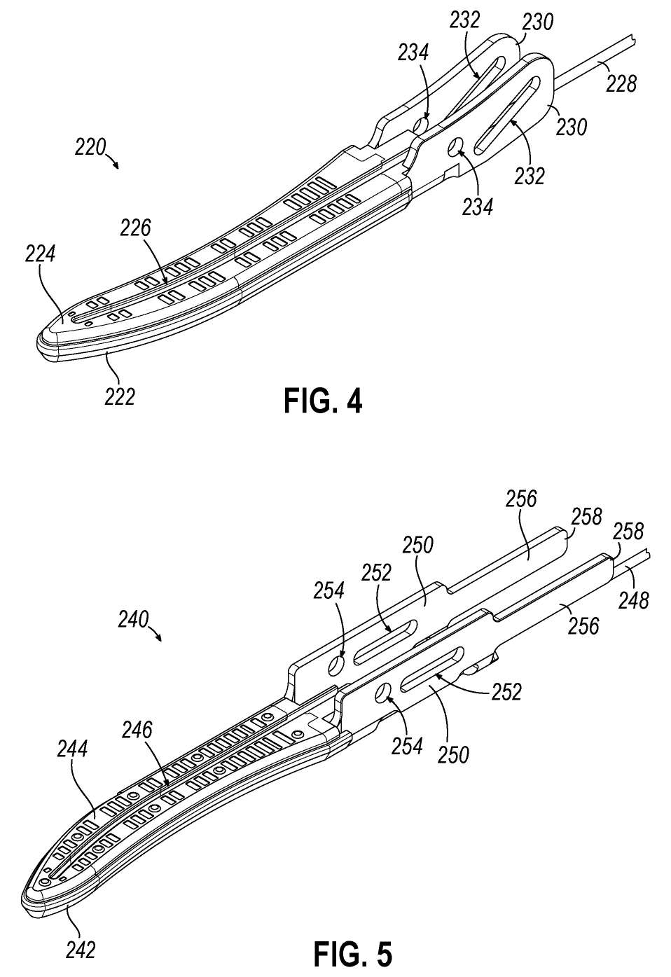

As shown in FIGS. 2A-4 jaw (220) includes a body (222) and an electrode (224). In versions where body (222) includes an electrically conductive material (e.g., metal, etc.), an electrically insulative material (e.g., polymer, etc.) may be interposed between body (222) and electrode (224) to electrically isolate the electrically conductive portion of body (222) relative to electrode (224). For instance, an insulative plastic material may be overmolded over a metallic portion of body (222) (or otherwise be secured to the metallic portion of body (222)), and electrode (224) may be secured to the other side of the plastic material, such that the plastic material is interposed between electrode (224) and the metallic portion of body (222). In the present example, body (222) and electrode (224) curve laterally away from a central longitudinal axis along a distal portion of the length of jaw (220). In some other versions, these features are substantially straight along the entire length of jaw (220).

Jaw (220) defines a knife slot (226) passing through electrode (224). Knife slot (226) is configured to accommodate longitudinal motion of knife (184) relative to jaw (220) while jaws (220, 240) are in the closed configuration. Jaw (220) further includes a pair of proximal flanges (230) which are laterally spaced apart from each other. Each flange (230) defines an elongate slot (232) and a pin opening (234). Each elongate slot (232) is oriented obliquely along its corresponding flange (230), which provides closure motion to jaw (220) as described in greater detail below. A wire (228) extends proximally from jaw (220). Wire (228) is coupled with electrode (224) and extends along the length of shaft assembly (100) as described in greater detail below. Wire (228) is further configured to couple with a source of RF energy (e.g., an external generator), such that wire (228) is configured to provide RF energy to electrode (224).

As shown in FIGS. 2A-3 and 5 jaw (240) includes a body (242) and an electrode (244). In versions where body (242) includes an electrically conductive material (e.g., metal, etc.), an electrically insulative material (e.g., polymer, etc.) may be interposed between body (242) and electrode (244) to electrically isolate the electrically conductive portion of body (242) relative to electrode (244). For instance, an insulative plastic material may be overmolded over a metallic portion of body (242) (or otherwise be secured to the metallic portion of body (242)), and electrode (244) may be secured to the other side of the plastic material, such that the plastic material is interposed between electrode (244) and the metallic portion of body (242). In the present example, body (242) and electrode (244) curve laterally away from a central longitudinal axis along a distal portion of the length of jaw (240). In some other versions, these features are substantially straight along the entire length of jaw (240).

Jaw (240) defines a knife slot (246) passing through electrode (244). Knife slot (246) is configured to accommodate longitudinal motion of knife (144) relative to jaw (240). Jaw (240) further includes a pair of proximal flanges (250) which are laterally spaced apart from each other. Each flange (250) defines an elongate slot (252) and a pin opening (254). Each elongate slot (252) is oriented longitudinally along its corresponding flange (250). A beam (256) extends proximally from each flange (250) and terminates in a respective proximal face (258). A wire (248) extends proximally from jaw (240). Wire (248) is coupled with electrode (244) and extends along the length of shaft assembly (100) as described in greater detail below. Wire (248) is further configured to couple with a source of RF energy (e.g., an external generator), such that wire (248) is configured to provide RF energy to electrode (244).

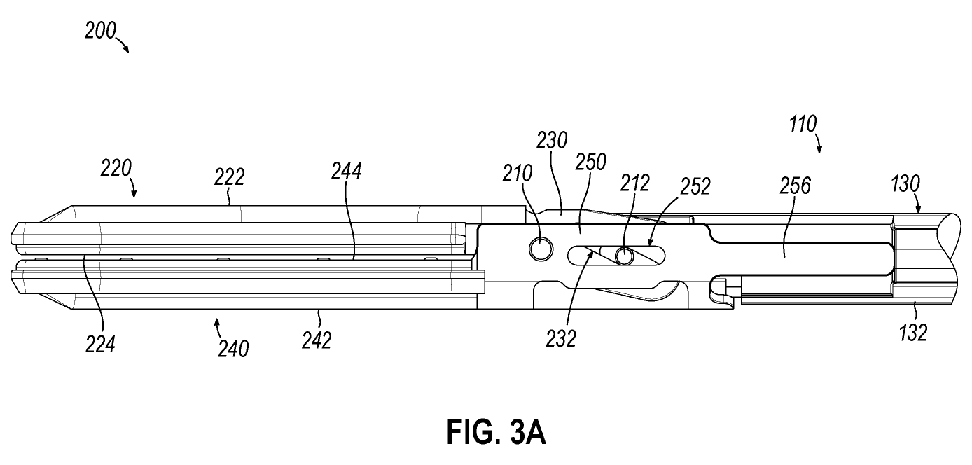

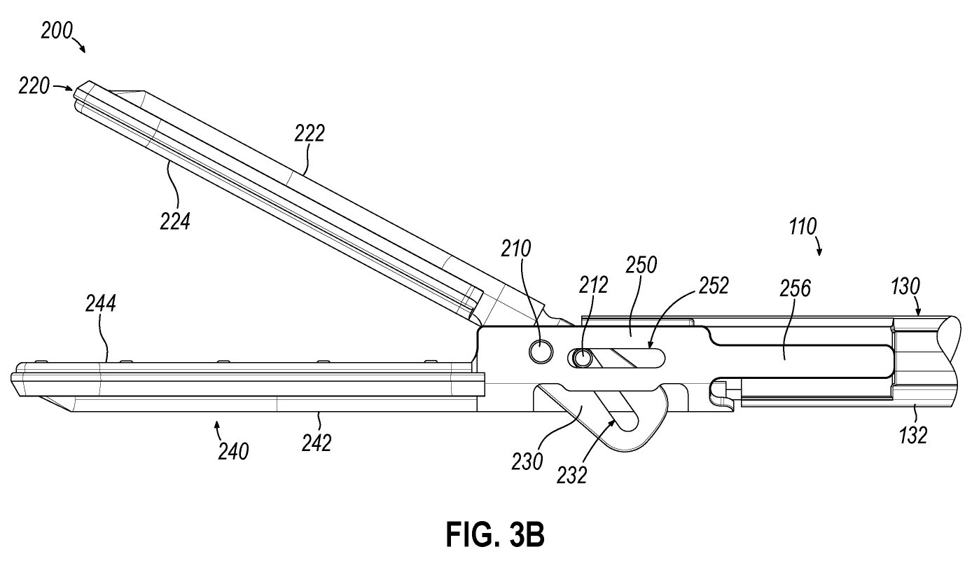

As shown in FIGS. 3A-3B, a pin (210) is disposed through openings (234, 254) of flanges (230, 250) of jaws (220, 240), thereby pivotably coupling jaws (220, 240) to each other. Another pin (212) is disposed through slots (232, 252). Pin (212) is operable to be driven between a proximal position (FIG. 3A) and a distal position (FIG. 3B) as will be described in greater detail below. Because slots (232) of flanges (230) are oriented obliquely while slots (252) of flanges (250) are oriented longitudinally in this example, longitudinal motion of pin (212) along slots (232, 252) will drive pivotal motion of jaws (220, 240). In particular, pin (212) will drive jaw (220) to an open position when pin (212) is in a proximal position (FIG. 3A); and to a closed position when pin (212) is in a distal position (FIG. 3B). Alternatively, jaws (220, 240) may be driven between open and closed configurations using any other suitable features or techniques.

In use, tissue (e.g., a vessel, etc.) may be positioned between jaws (220, 240) while jaws (220, 240) are in an open configuration. Then, jaws (220, 240) may be driven to the closed configuration to clamp the tissue. While jaws (220, 240) remain closed, knife (184) may be driven from the proximal position to the distal position to sever the tissue. In addition, bipolar RF energy may be applied to the tissue via electrodes (224, 244) to thereby seal the tissue. In some versions, electrode (244) serves as an active electrode while electrode (224) serves as a return electrode. Alternatively, this relationship may be reversed. In some examples of use, bipolar RF energy may be applied to the tissue via electrodes (224, 226) without knife (184) being advanced distally through the tissue. In either scenario, jaws (220, 240) may be returned to the open configuration to release the tissue when appropriate.

### B. Overview of Shaft Assembly

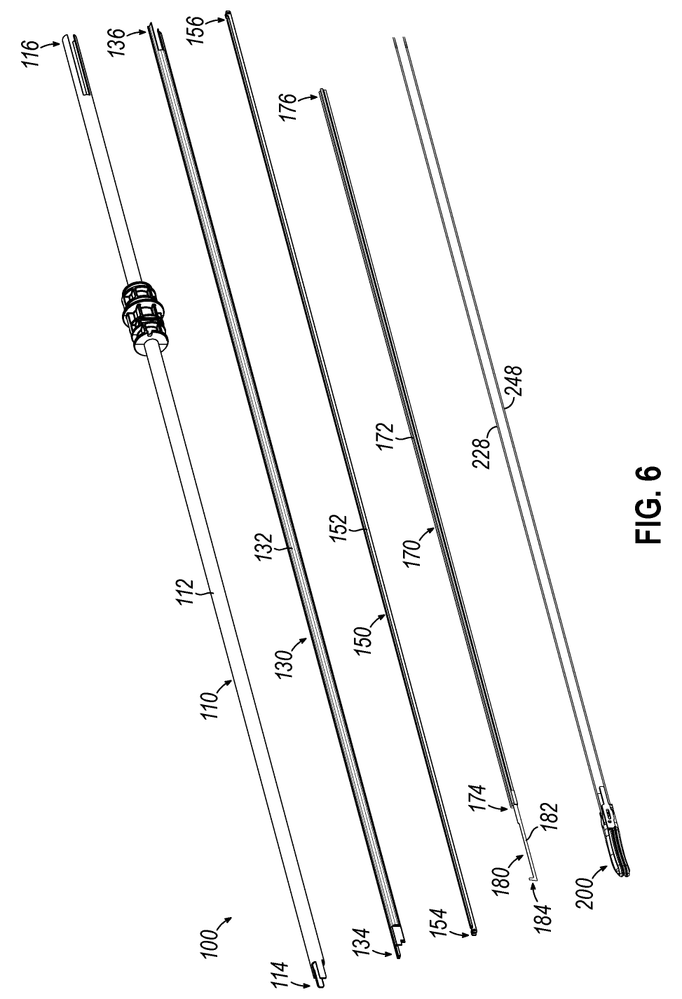

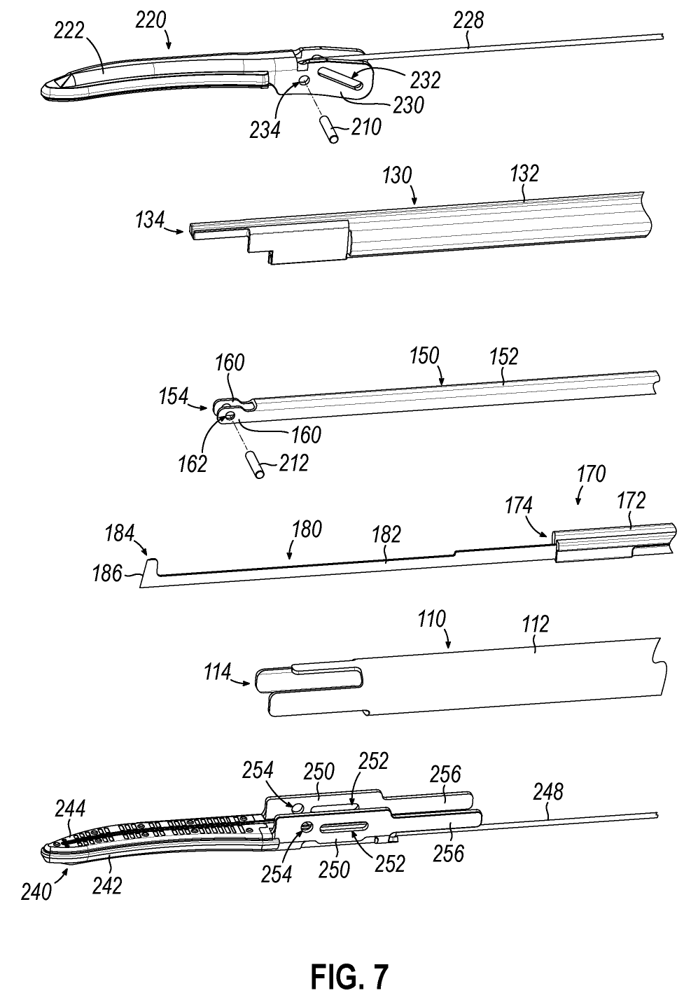

FIGS. 6-7 show components of shaft assembly (100) in greater detail. As shown, shaft assembly (100) of this example includes an outer shaft (110), an inner guide (130), a closure beam (150), and a knife assembly (170). Outer shaft (110) of this example includes a hollow tubular body (112) with a distal end (114) and a proximal end (116). End effector (200) is positioned at distal end (114) while proximal end (116) is disposed in body (20). Inner guide (130) of this example includes an elongate body (132) with a distal end (134) and a proximal end (136). Elongate body (132) of inner guide (130) is disposed within tubular body (112) of outer shaft (110). Elongate body (132) is laterally offset relative to the central longitudinal axis of tubular body (112) (i.e., not centered along the central longitudinal axis of tubular body (112)). Elongate body (132) of inner guide (130) remains stationary relative to tubular body (112) of outer shaft (110) during operation of instrument (10) in the present example. End effector (200) is positioned at distal end (134) while proximal end (136) is disposed in body (20). Wires (228, 248) are interposed between elongate body (132) of inner guide (130) and the inner surface of tubular body (112) of outer shaft (110) in this example.

Closure beam (150) of this example includes an elongate body (152) with a distal end (154) and a proximal end (156). Elongate body (152) of closure beam (150) is disposed within tubular body (112) of outer shaft (110), laterally adjacent to elongate body (132) of inner guide (130). In the present example, elongate body (152) is radially centered along the central longitudinal axis of tubular body (112). Elongate body (152) of the present example has an upside-down “U” shaped cross-sectional profile, though elongate body (152) may alternatively have any other suitable cross-sectional profile. Proximal end (156) is disposed within body (20) and is coupled with jaw closure assembly (22), such that jaw closure assembly (22) is operable to drive longitudinal translation of closure beam (150) relative to outer shaft (110) and relative to inner guide (130). Distal end (154) includes a clevis arrangement formed by two flanges (160), each flange (160) having a respective pin opening (162). Pin (212) is disposed in openings (162), in addition to being disposed in slots (232, 252) of flanges (230, 250) of jaws (220, 240). Thus, closure beam (150) drives translation of pin (212) to thereby drive opening and closing of jaws (220, 240) as described above. Alternatively, any other suitable components and arrangements may be used to drive opening and closing of jaws (220, 240).

Knife assembly (170) of the present example includes an elongate body (172) and a knife beam (180). Knife beam (180) is fixedly secured relative to elongate body (172) and extends distally relative to a distal end (174) of elongate body (172). Knife beam (180) includes a beam body (182) that distally terminates in knife (184). Knife (184) includes an upright portion (186) having a distal cutting edge (188). Elongate body (172) of knife assembly (170) is disposed within tubular body (112) of outer shaft (110), laterally adjacent to elongate body (132) of inner guide (130) and elongate body (152) of closure beam (150). Elongate body (172) is laterally offset relative to the central longitudinal axis of tubular body (112) (i.e., not centered along the central longitudinal axis of tubular body (112)), though the central longitudinal axis of tubular body (112) extends along a portion of elongate body (172) in this example. Elongate body (172) of the present example has a “U” shaped cross-sectional profile, though elongate body (152) may alternatively have any other suitable cross-sectional profile. In the present example, the “U” shape of elongate body (172) is partially nested within the “U” shape of elongate body (152).

A proximal end (176) of elongate body (172) is disposed within body (20) and is coupled with knife drive assembly (24), such that knife drive assembly (24) is operable to drive longitudinal translation of knife assembly (170) relative to outer shaft (110) and relative to inner guide (130). In the present example, knife assembly (170) and closure beam (150) are translatable independently relative to each other. Also in the present example, knife assembly (170) and knife drive assembly (24) are configured such that when knife (184) is at the distal-most position of the range of motion of knife (184) cutting edge (188) is spaced proximally from the distal ends of knife slots (226, 246). In other words, cutting edge (188) will not contact jaws (220, 240) at the distal ends of knife slots (226, 246) when knife (184) reaches the distal-most position of the range of motion of knife (184). This may prevent premature dulling of cutting edge (188) that might otherwise occur if cutting edge (188) were allowed to contact jaws (220, 240) at the distal ends of knife slots (226, 246) when knife (184) reaches the distal-most position of the range of motion of knife (184). In some other versions, knife assembly (170) and knife drive assembly (24) are configured to allow cutting edge (188) to contact jaws (220, 240) at the distal ends of knife slots (226, 246) when knife (184) reaches the distal-most position of the range of motion of knife (184).

### II. Examples of Features to Promote Consistency in Manufacture and Performance

As noted above, it may be desirable to minimize manufacturing variations and associated tolerances. In the context of instrument (10), this may include ensuring that the fit between end effector (200) and shaft assembly (100) is as consistent and predictable as possible. This may reduce the risk of undesirable, unintended movement of one or more components of end effector (200) relative to shaft assembly (100) during use of instrument (10). Similarly, it may be desirable to promote consistency and precision in the range of intentional motion of one or more components of instrument (10), such as the longitudinal range of travel for knife assembly (170). As another example, it may be desirable to ensure that end effector (200) and shaft assembly (100) are consistently and precisely positioned relative to each other before end effector (200) is fixed in place relative to shaft assembly (100). The following describes examples of features that may be included in end effector (200) and shaft assembly (100) to promote consistency in manufacture, minimizing variations and tolerances, which would in turn promote consistency in performance of instrument (10) during use.

### A. Examples of Features to Predictably Prevent and Arrest Relative Motion between Components

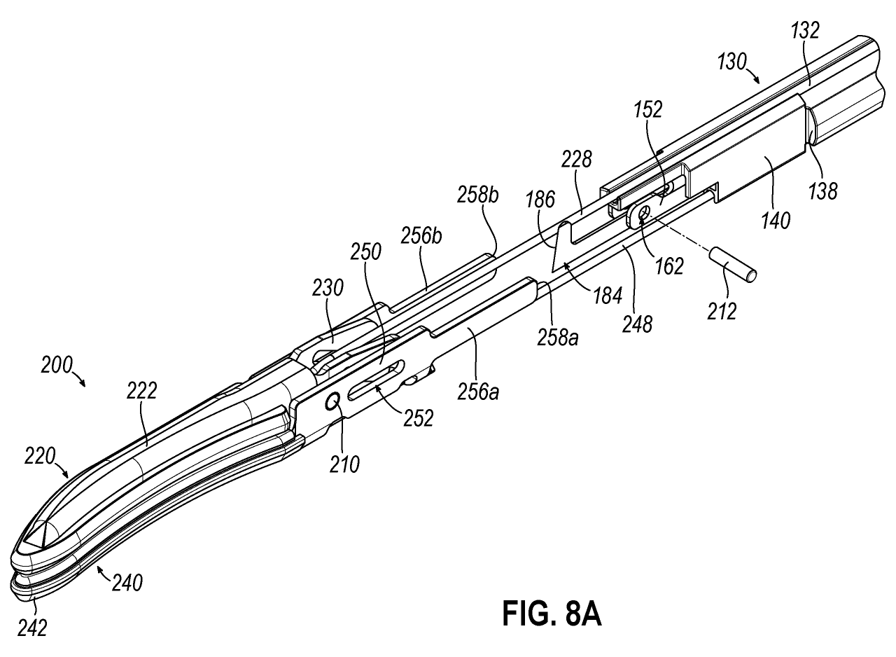

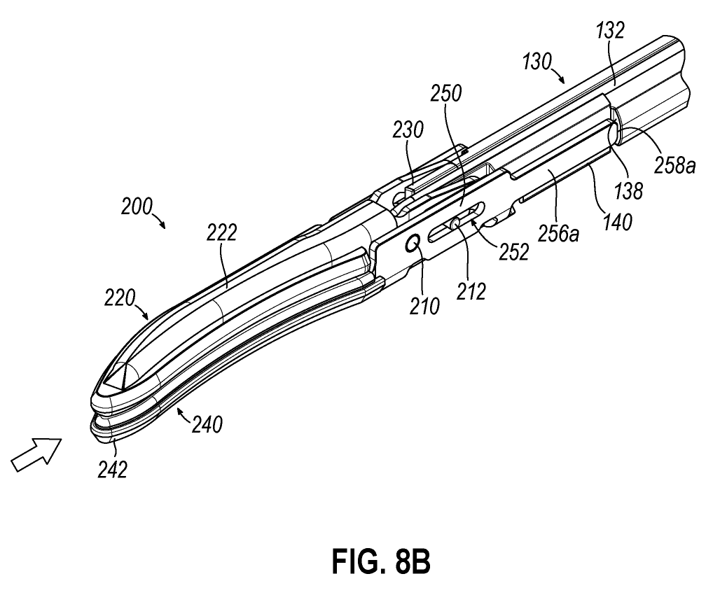

FIGS. 8A-8B show components of end effector (200) and shaft assembly (100) that interact with each other to predictably prevent unintended relative motion between components on a consistent basis. Outer shaft (110) is omitted from FIGS. 8A-8B for clarity. As noted above, jaw (240) of end effector (200) includes a pair of proximal flanges (250), with a beam (256) extending proximally from each flange (256). Beams (256) are flat, planar structures (e.g., formed by a stamping process) in this example. Each beam (256) terminates in a respective proximal face (258). Beams (256) may be separately considered as a first beam (256a) and a second beam (256b), with a respective first proximal face (258a) and second proximal face (258b). First beam (256a) interacts with complementary features of inner guide (130) to consistently position jaw (240) relative to inner guide (130) and to maintain such relative positioning throughout operation of instrument (10). As best seen in FIG. 8A, these features of inner guide (130) include a flat, laterally facing surface (140) near the distal end of elongate body (132); and a distally facing surface (138) positioned near the proximal end of laterally facing surface (140).

As shown in the transition between FIG. 8A and FIG. 8B, as end effector (200) is moved proximally toward shaft assembly (100) during manufacture of instrument (10), first beam (256a) slides along surface (140) until first proximal face (258a) abuts surface (138). This engagement between proximal face (258a) and surface (138) arrests further proximal motion of end effector (200) relative to shaft assembly (100). End effector (200) is then fixedly secured to shaft assembly (100) while proximal face (258a) abuts surface (138). The engagement between proximal face (258a) and surface (138) thus ensures that end effector (200) is at the appropriate longitudinal position relative to at least inner guide (130) when instrument (10) is assembled and throughout subsequent use of instrument (10).

Moreover, engagement between first beam (256a) and surface (140) prevents jaw (240) from rotating relative to inner guide (130) about the central longitudinal axis of shaft assembly (100). Engagement between first beam (256a) and surface (140) thus ensures that end effector (200) is at the appropriate angular position relative to at least inner guide (130) when instrument (10) is assembled and throughout subsequent use of instrument (10). To the extent that there is any slight angular misalignment of jaw (240) relative to inner guide (130) as end effector (200) is moved proximally into position relative to shaft assembly (100) during the transition shown in FIGS. 8A-8B, first beam (256a) and surface (140) may cooperate to provide guidance to jaw (240) into the appropriate angular position. In other words, first beam (256a) may provide an aspect of self-piloting or self-guidance to jaw (240) during manufacture of instrument (10).

As described above, proximal face (258a) and surface (138) cooperate to establish (during manufacture) and maintain (during use) appropriate longitudinal positioning of jaw (240) relative to inner guide (130). These same features may also cooperate to establish (during manufacture) and maintain (during use) appropriate longitudinal positioning of inner guide (130) relative to outer shaft (110), relative to other components of shaft assembly (100), relative to end effector (200), and/or relative to other components of instrument (10). As described in greater detail below, beams (256) are fixedly secured to outer shaft (110) during the manufacture of instrument (10). Thus, the longitudinal structural and spatial relationship provided between jaw (240) and inner guide (130) will be effectively transferred to outer shaft (110) and other components to which jaw (240) is fixedly secured. In other words, the longitudinal structural and spatial relationship between inner guide (130) and outer shaft (110), etc., will be established and fixedly maintained due to the combination of: (i) engagement between proximal face (258a) and surface (138), and (ii) the fixed securement between beams (256) and outer shaft (110).

As also described above, first beam (256a) and surface (140) cooperate to establish (during manufacture) and maintain (during use) appropriate angular positioning of jaw (240) relative to inner guide (130). These same features may also cooperate to establish (during manufacture) and maintain (during use) appropriate angular positioning of inner guide (130) relative to outer shaft (110), relative to other components of shaft assembly (100), relative to end effector (200), and/or relative to other components of instrument (10). As already noted, beams (256) are fixedly secured to outer shaft (110) during the manufacture of instrument (10). Thus, the angular structural and spatial relationship provided between jaw (240) and inner guide (130) will be effectively transferred to outer shaft (110) and other components to which jaw (240) is fixedly secured. In other words, the angular structural and spatial relationship between inner guide (130) and outer shaft (110), etc., will be established and fixedly maintained due to the combination of: (i) engagement between first beam (256a) and surface (140), and (ii) the fixed securement between beams (256) and outer shaft (110).

While the foregoing describes engagement between first beam (256a) and surface (140) as establishing and maintaining appropriate angular positioning of jaw (240) relative to inner guide (130), and as establishing and maintaining appropriate angular positioning of inner guide (130) relative to outer shaft (110), etc., there may be similar engagement between second beam (256b) and one or more complementary surfaces of elongate body (132). Such complementary surface(s) of elongate body (132) may be on the other side of elongate body (132) relative to surface (140).

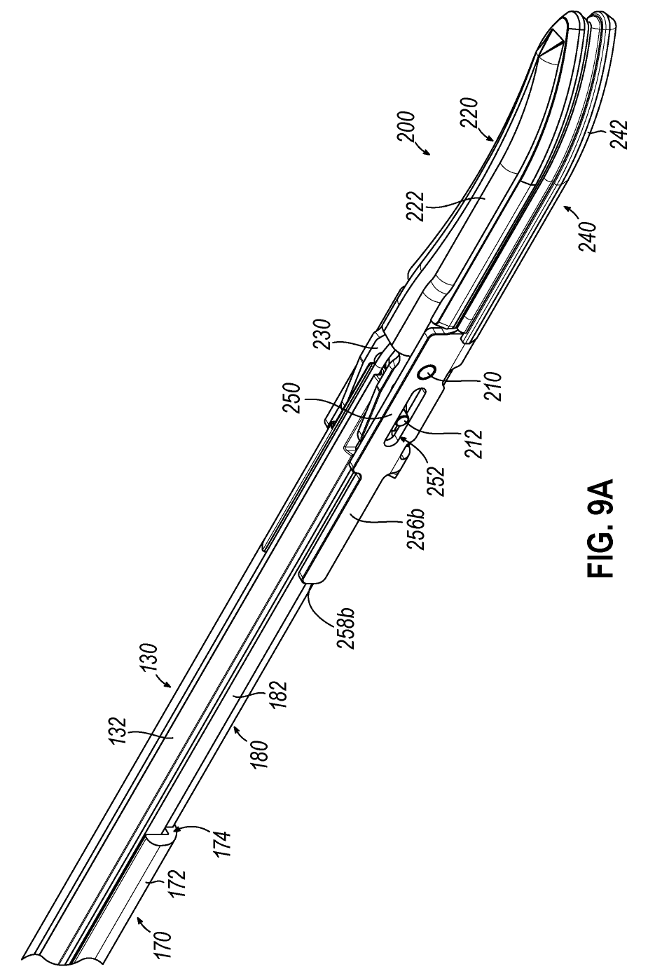

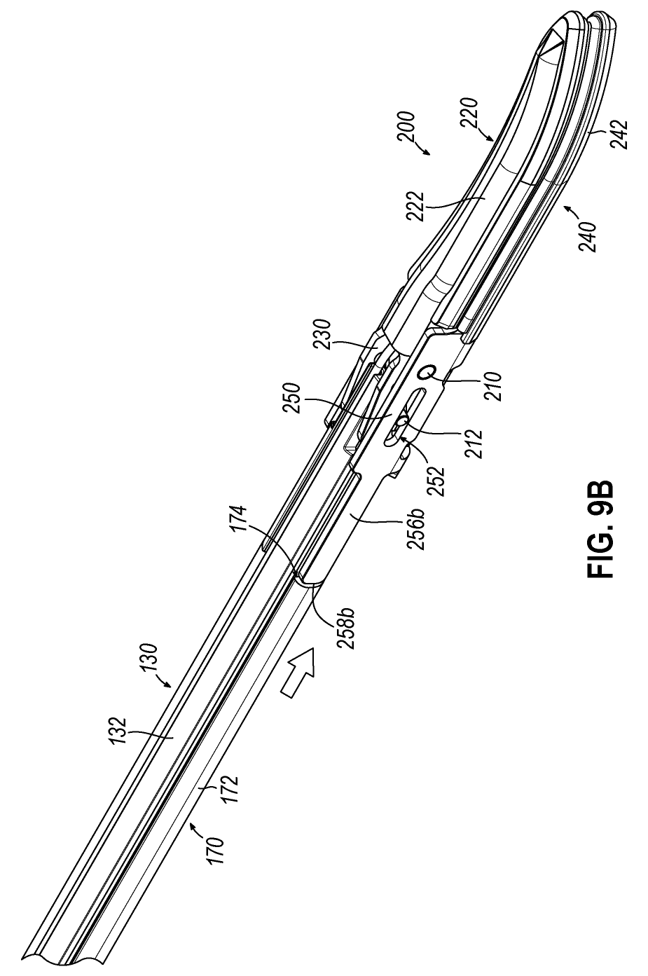

FIGS. 9A-9B show additional components of end effector (200) and shaft assembly (100) that interact with each other to predictably arrest intended relative motion between components on a consistent basis. Outer shaft (110) is omitted from FIGS. 9A-9B for clarity. FIG. 9A shows end effector (200) and shaft assembly (100) in a stage of operation where knife assembly (170) is in a proximal position. As shown, distal end (174) of elongate body (172) is proximally positioned relative to second proximal face (258b) at this stage of operation.

When knife assembly (170) is advanced distally by knife drive assembly (24) to the distal position shown in FIG. 9B, distal end (174) of elongate body (172) eventually contacts second proximal face (258b). Second proximal face (258b) thus provides a hard stop arresting further distal advancement of knife assembly (170). In the present example, these components are arranged such that engagement between distal end (174) and second proximal face (258b) will arrest distal advancement of knife assembly (170) before cutting edge (188) of knife (184) contacts jaws (220, 240) at the distal ends of knife slots (226, 246). Engagement between distal end (174) and second proximal face (258b) will thus prevent premature dulling of cutting edge (188) that might otherwise occur if cutting edge (188) were allowed to contact jaws (220, 240) at the distal ends of knife slots (226, 246) when knife (184) reaches the distal-most position of the range of motion of knife (184). It should be understood that the surface areas of distal end (174) and second proximal face (258b) may be relatively large compared to hard stop features in some conventional instruments, and these larger surface areas may provide enhanced reliability for the hard stop functionality.

As described above, while first beam (256a) and second beam (256b) have similar structural configurations and are simply diametrically opposed to each other in the present example, first beam (256a) and second beam (256b) may provide different respective functionalities during the manufacture and subsequent use of instrument (10). While first beam (256a) and second beam (256b) are the same length in the present example, there may be other versions where the length of first beam (256a) is different from the length of second beam (256b). Some other versions may also provide other structural differences between first beam (256a) and second beam (256b).

### B. Examples of Features to Promote Consistent Relative Positioning between Components Before Fixation of Components

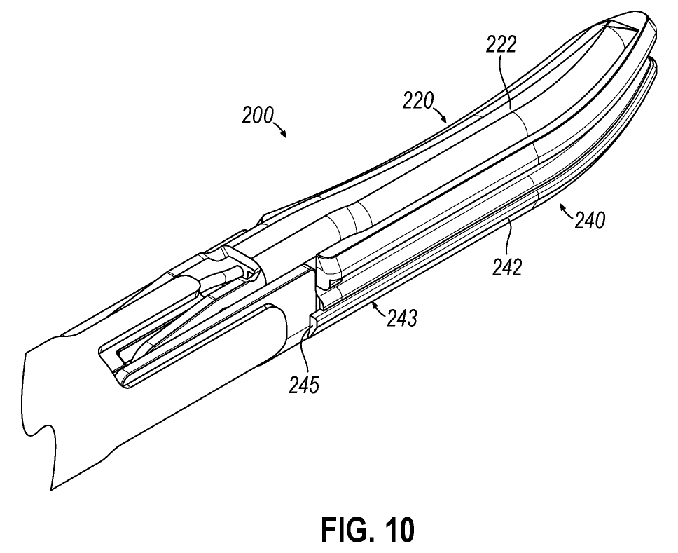

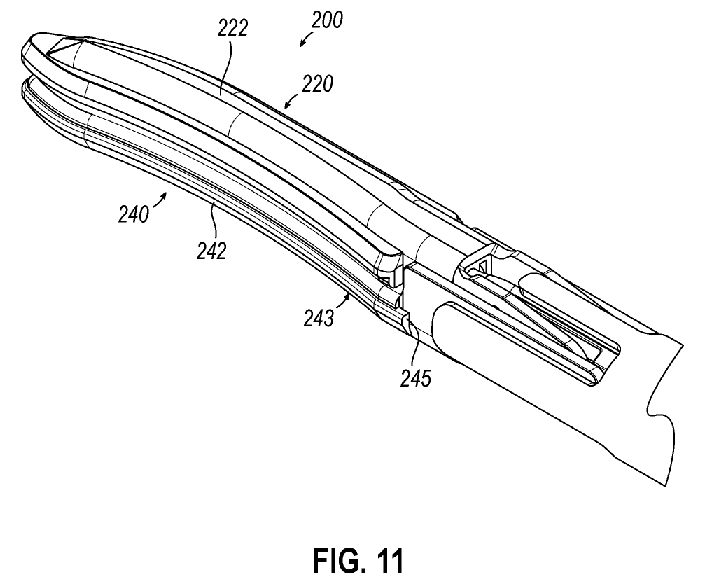

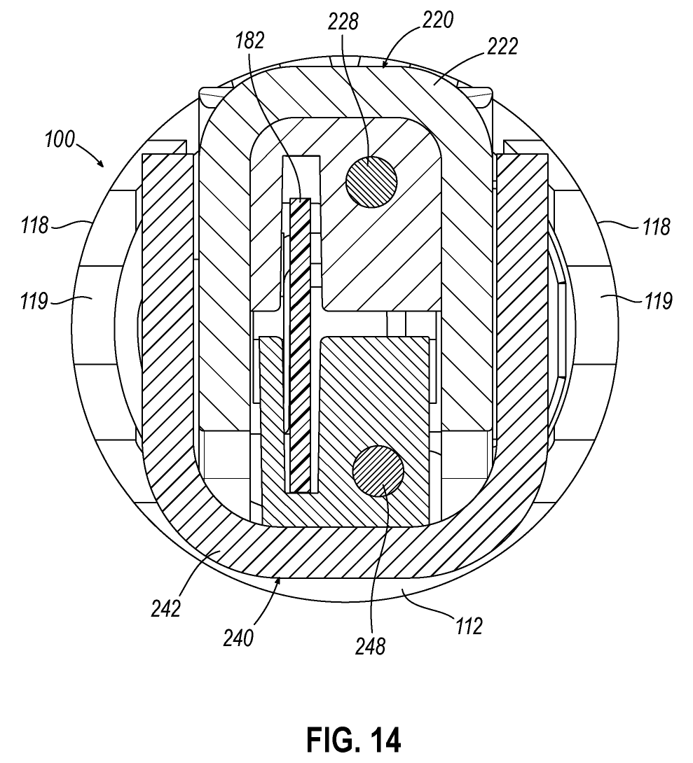

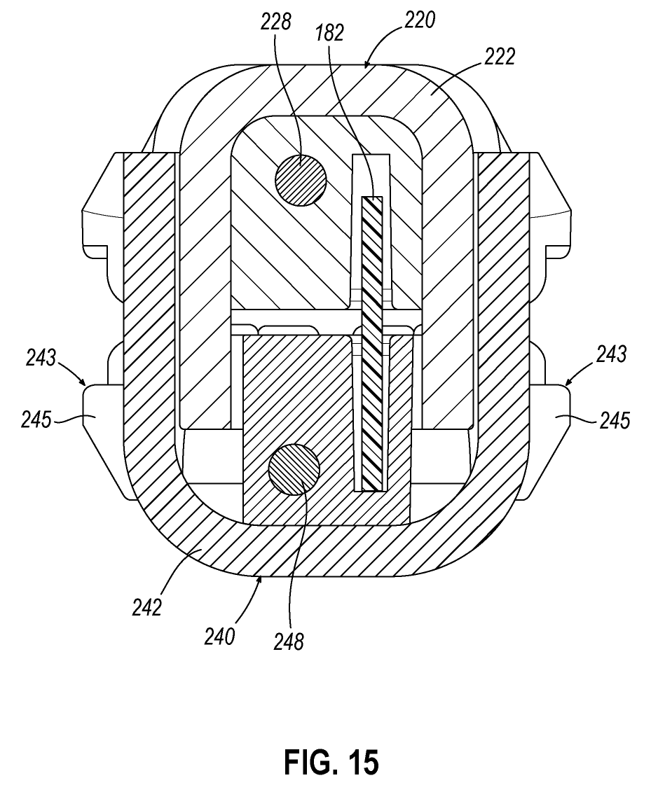

FIGS. 10-15 show components of end effector (200) and shaft assembly (100) providing datum features may be used to promote consistent relative positioning between end effector (200) and shaft assembly (100) before end effector (200) is secured to shaft assembly (100) during a process of manufacturing instrument (10). As best seen in FIGS. 10-13 and 15, jaw (240) includes a rib (243) outwardly extending laterally outwardly relative to the rest of body (242). In some versions, rib (243) is provided by a plastic overmold formed about body (242), though rib (243) may be otherwise constructed. Rib (243) wraps around the front of jaw (240) and proximally terminates at or near each flange (250). A pair of proximally facing surfaces (245) are defined at each proximal end of rib (243). In the present example, rib (243) extends continuously from one proximally facing surface (245) to the other proximally facing surface (245), wrapping around the distal end of jaw (240). In some other versions, jaw (240) has two (or more) ribs (243), with each proximally facing surface (245) being positioned at a proximal end of a respective, separate rib (243).

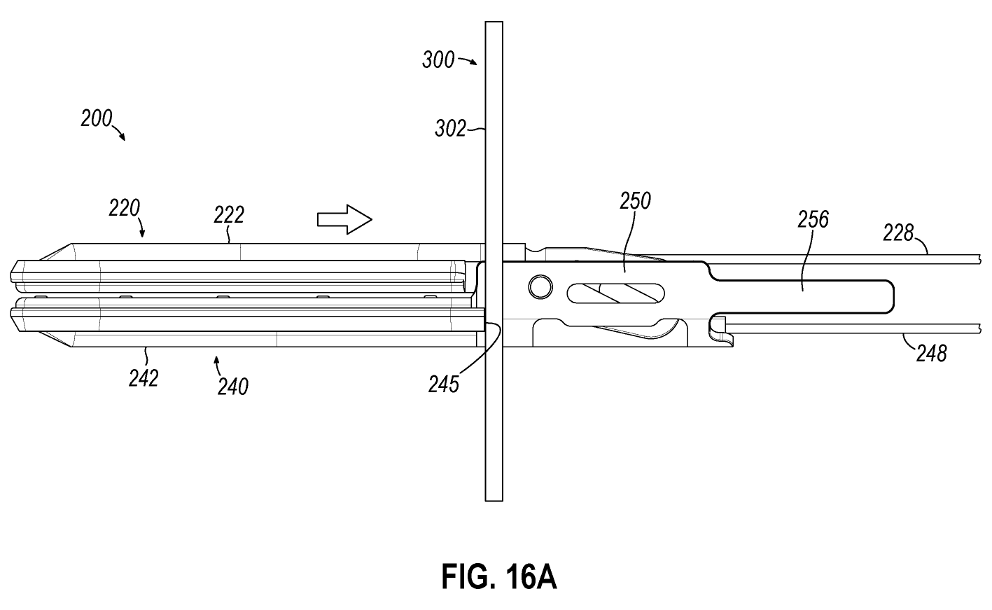

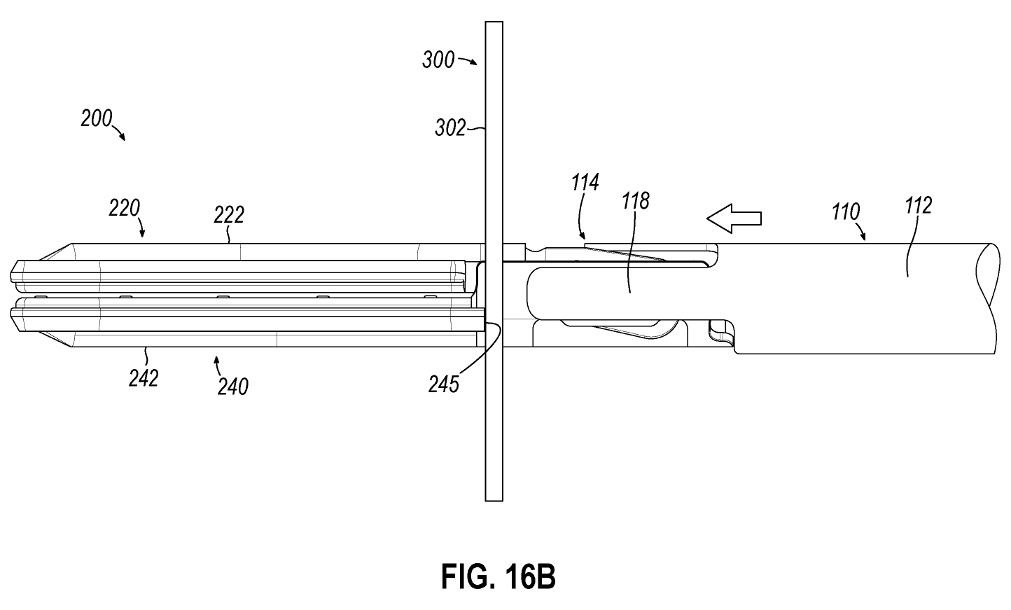

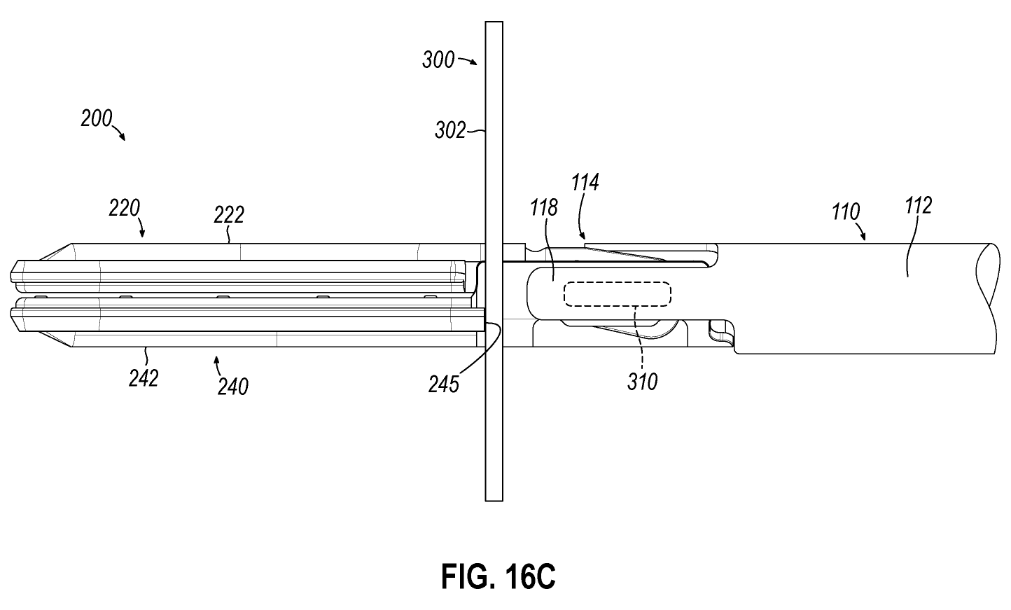

The proximally facing surfaces (245) at the proximal ends of rib (243) are configured to engage a fixture, and thereby provide and maintain a predetermined position of end effector (200), during a stage of the process of manufacturing instrument (10) where end effector (200) is secured to shaft assembly (100). An example of this stage of the manufacturing process is shown in FIGS. 16A-16C. As shown in FIG. 16A, surfaces (245) are urged into engagement with a distal face (302) of a fixture (300) during a first sub-stage of this process. While the motion arrow in FIG. 16A shows end effector (200) being moved proximally to engage fixture (300), some versions may provide distal movement of fixture (300) to engage end effector (200). It should also be understood that fixture (300) is represented schematically in FIGS. 16A-16C and that fixture (300) may take any suitable form.

With surfaces (245) of ribs (243) engaging surface (302) of fixture (300), shaft assembly (100) is moved into place as shown in FIG. 16B. Surfaces (245) remain in engagement with surface (302) during this stage; and in some cases this engagement is maintained through a predetermined force where at least one of surfaces (245, 302) is pressed against the other of surfaces (245, 302). While not shown in FIG. 16B, it should be understood that first beam (256a) may slide along surface (140) of inner guide (130), as described above, during the transition from the state shown in FIG. 16A to the state shown in FIG. 16B. It should also be understood that first proximal face (258a) will eventually abut surface (138) of inner guide (130), as described above, during the transition from the state shown in FIG. 16A to the state shown in FIG. 16B. In some versions, the engagement between the above-noted features of first beam (256a) and inner guide (130) is maintained through a predetermined force where inner guide (130) (and/or other components of shaft assembly (100)) is pressed distally against first proximal face (258a).

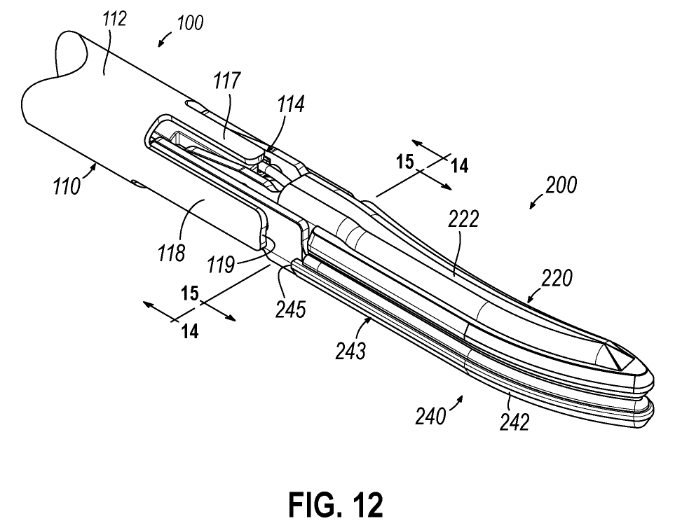

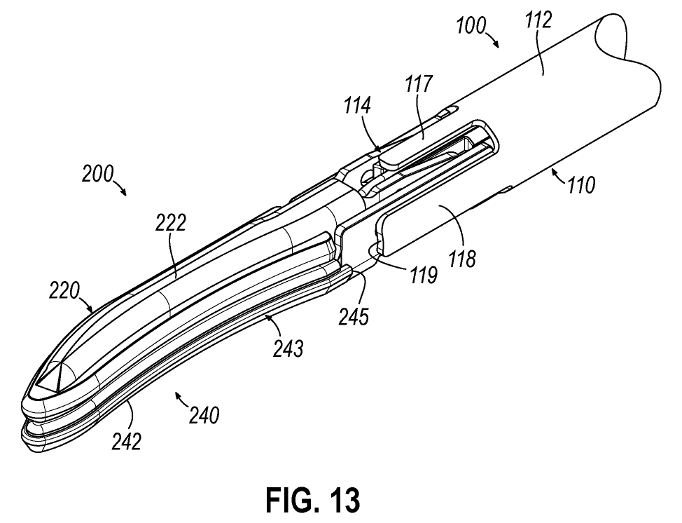

With shaft assembly (100) appropriately positioned in relation to end effector (200), shaft assembly (100) is then fixedly secured to end effector (200). In the present example, this securement is achieved via welding. In the example shown in FIG. 16C, a weld (310) is provided to directly secure a side projection (118) at distal end (114) of outer shaft (110) to first beam (256a). Also in the present example, but not shown in FIG. 16C, a similar weld is provided to directly secure an opposing side projection (118) at distal end of outer shaft (110) to second beam (256b). While welding is used in the present example to fixedly secure shaft assembly (100) to end effector (200) via outer shaft (110), other techniques and/or other components may be used to fixedly secure shaft assembly (100) to end effector (200). In the present example, an upper projection (117) is also provided at distal end (114) of outer shaft (110) (see FIGS. 12-13), though upper projection (117) is not used to secure shaft assembly (100) to end effector (200). Upper projection (117) may provide shielding to moving components at the proximal end of end effector (200). In some versions, upper projection (117) may provide other functionalities.

FIGS. 12-14 show components of shaft assembly (100) providing datum features may be used to promote consistent relative positioning between end shaft assembly and body (20) before body (20) is secured to shaft assembly (100) during a process of manufacturing instrument (10). These features include distally facing surfaces (119) of side projections (118) that project distally from outer tube (112).

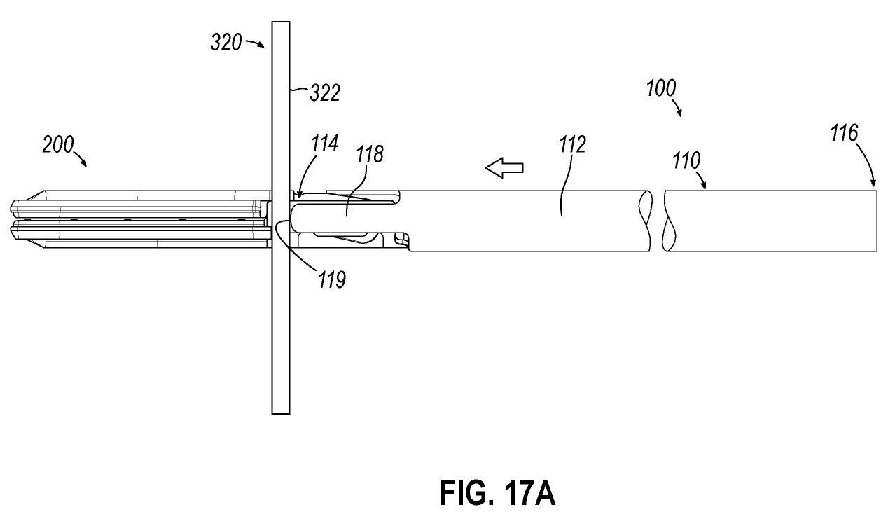

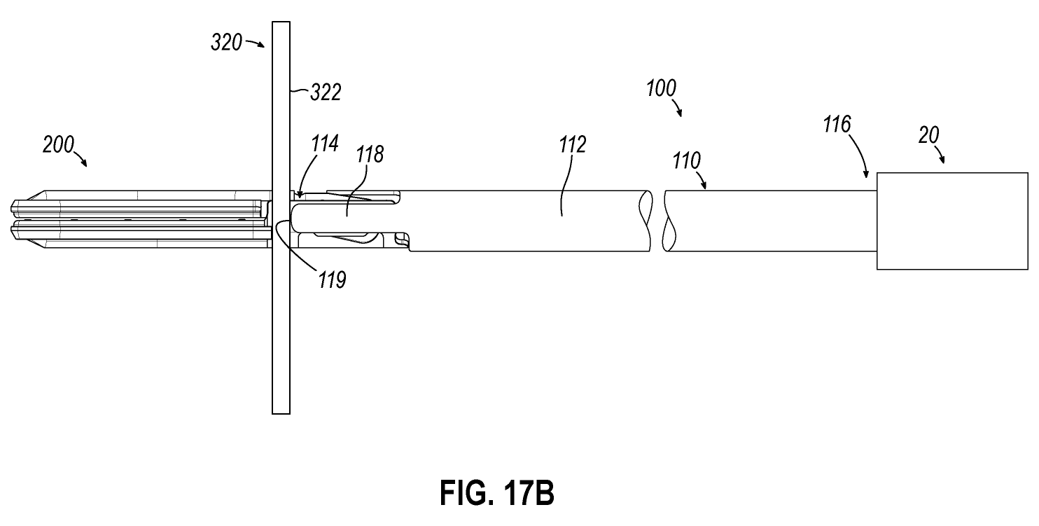

These distally facing surfaces (119) at the distal end of each side projection (118) are configured to engage a fixture, and thereby provide and maintain a predetermined position of end shaft assembly (100), during a stage of the process of manufacturing instrument (10) where body (20) is secured to shaft assembly (100). An example of this stage of the manufacturing process is shown in FIGS. 17A-17B. As shown in FIG. 17A, surfaces (119) are urged into engagement with a proximal face (322) of a fixture (320) during a first sub-stage of this process. While the motion arrow in FIG. 17A shows shaft assembly (100) being moved distally to engage fixture (320), some versions may provide proximal movement of fixture (320) to engage shaft assembly (100). It should also be understood that fixture (320) is represented schematically in FIGS. 17A-17B and that fixture (320) may take any suitable form. While fixture (300) is not shown in FIGS. 17A-17B, some versions of the method of manufacturing instrument (10) may provide continued engagement between end effector (200) and fixture (300) during one or more of the sub-stages shown in FIGS. 17A-17B.

With distally facing surfaces (119) of side projections (118) engaging surface (322) of fixture (320), body (20) is moved into place as shown in FIG. 17B. Surfaces (119) remain in engagement with surface (322) during this stage; and in some cases this engagement is maintained through a predetermined force where at least one of surfaces (119, 322) is pressed against the other of surfaces (119, 322). With body (20) appropriately positioned in relation to shaft assembly (100), body (20) is then fixedly secured to shaft assembly (100). Such securement may be achieved in any suitable fashion, including but not limited to welding, snap-fitting, use of one or more fasteners (e.g., pins, screws, rivets, etc.), and/or use of any other components or techniques, including combinations thereof.

While fixture (320) is not introduced until after shaft assembly (100) has been fixedly secured to end effector (200) in the example provided above, some other versions may provide the engagement between shaft assembly (100) and fixture (320), as shown in FIG. 17A, at the sub-stage shown in FIG. 16B (i.e., before shaft assembly (100) has been fixedly secured to end effector (200)). Similarly, some versions may provide the engagement between shaft assembly (100) and fixture (320), as shown in FIG. 17A, at or before the sub-stage shown in FIG. 16A. In some versions where shaft assembly (100) is engaged with fixture (320) before end effector (200) is engaged with fixture (300), end effector (200) may be moved proximally into engagement with fixture (300) while fixtures (300, 320) remain fixed relative to each other and while shaft assembly (100) remains engaged with fixture (320).

After the above-described sub-stages of manufacture are performed, instrument (10) may be removed from fixtures (300, 320). As noted above, some versions may provide removal of end effector (200) and shaft assembly (100) from fixture (300) before the sub-stages of manufacture illustrated in FIGS. 17A-17B are performed.

In the examples provided above, outwardly projecting features in the form of surfaces (245) of ribs (243) and surfaces (119) of side projections (118) are used to provide datum features. This may provide advantages over some versions of instruments where recesses or other inwardly projecting features provide datum features. For instance, such recesses or other inwardly projecting features providing datum features may impinge on the interior space within shaft assembly (100), which may in turn reduce the amount of space that is left for interior components of shaft assembly (100). This may in turn reduce the robustness of shaft assembly (100) and/or increase the risk of interior components snagging within shaft assembly (100). Similarly, recesses or other inwardly projecting features providing datum features may impinge on the interior space within end effector (200), which may in turn reduce the amount of space that is left for interior components of end effector (200) and/or increase the risk of interior components snagging within end effector (200). This may in turn reduce the robustness of end effector (200). Thus, the outwardly projecting datum features of instrument (10) may avoid such compromises to the interior space within shaft assembly (100) and end effector (200); and may thus avoid compromises to the robustness of shaft assembly (100) and end effector (200) and/or avoid risks of interior components snagging within shaft assembly (100) and end effector (200).

### III. Examples of Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

Example 1

An apparatus comprising: (a) body; (b) a shaft assembly defining a longitudinal axis, the shaft assembly including an inner guide, the inner guide including a laterally facing surface and a distally facing surface; and (c) an end effector, the end effector including: (i) a first jaw, and (ii) a second jaw, the first jaw being movable toward and away from the second jaw, the second jaw including a first proximally projecting beam, the first proximally projecting beam being positioned and configured to engage the laterally facing surface of the inner guide to thereby establish and maintain a predetermined angular position of the end effector relative to the shaft assembly, the first proximally projecting beam being further positioned and configured to engage the distally facing surface of the inner guide to thereby establish and maintain a predetermined longitudinal position of the end effector relative to the shaft assembly.

Example 2

The apparatus of Example 1, the distally facing surface of the inner guide being positioned at a proximal end of the laterally facing surface.

Example 3

The apparatus of any of Examples 1 through 2, the shaft assembly further including an outer tube, the inner guide being disposed within the outer tube, first proximally projecting beam being radially interposed between the inner guide and an inner surface of the outer tube, the inner guide being radially interposed between the first proximally projecting beam and the longitudinal axis.

Example 4

The apparatus of any of Examples 1 through 3, the inner guide being laterally offset relative to the longitudinal axis.

Example 5

The apparatus of any of Examples 1 through 4, the shaft assembly further including a knife assembly having a knife, the knife being movable through one or both of the first jaw or the second jaw.

Example 6

The apparatus of Example 5, the knife assembly further including a body extending through the shaft assembly, the body of the knife assembly being translatable relative to the inner guide between a proximal position and a distal position.

Example 7

The apparatus of Example 6, the body of the knife assembly having a distally facing surface, the second jaw including a second proximally projecting beam, the distally facing surface of the body of the knife assembly being configured to engage the second proximally projecting beam when body of the knife assembly is at the distal position.

Example 8

The apparatus of Example 7, the second jaw defining a knife slot having a distal end, the knife being translatable along the knife slot of the second jaw, the distally facing surface of the body of the knife and the second proximally projecting beam being configured and arranged to prevent the knife from reaching the distal end of the knife slot when the body of the knife assembly is in the distal position.

Example 9

The apparatus of any of Examples 7 through 8, the second proximally projecting beam being laterally positioned relative to the first proximally projecting beam on an opposite side of the longitudinal axis.

Example 10

The apparatus of any of Examples 7 through 9, the second proximally projecting beam being substantially parallel with the first proximally projecting beam.

Example 11

The apparatus of any of Examples 7 through 10, the first proximally projecting beam having a first length, the second proximally projecting beam having a second length, the second length being approximately equal to the first length.

Example 12

The apparatus of any of Examples 7 through 11, the shaft assembly further including an outer tube, the outer tube having a distal end with a first distally oriented side projection, and a second distally oriented side projection, the first distally oriented side projection being spaced apart from the second distally oriented side projection, the first distally oriented side projection being secured to the first proximally projecting beam, the second distally oriented side projection being secured to the second proximally projecting beam.

Example 13

The apparatus of Example 12, the first distally oriented side projection having an exposed first distally facing surface, the second distally oriented side projection having an exposed second distally facing surface, the first and second distally facing surfaces being positioned and configured to engage a proximally facing surface of a fixture.

Example 14

The apparatus of any of Examples 1 through 13, the second jaw including an outwardly extending rib, the rib having an exposed proximally facing surface, the proximally facing surface being positioned and configured to engage a distally facing surface of a fixture.

Example 15

The apparatus of any of Examples 1 through 14, the end effector further including one or more electrodes operable to apply radiofrequency energy to tissue.

Example 16

An apparatus comprising: (a) body; (b) a shaft assembly defining a longitudinal axis, the shaft assembly including; a knife assembly having a knife and a body, the knife being movable through one or both of the first jaw or the second jaw, the body of the knife assembly being translatable between a proximal position and a distal position, the body of the knife assembly having a distally facing surface; and (c) an end effector, the end effector including: (i) a first jaw, and (ii) a second jaw, the first jaw being movable toward and away from the second jaw, the second jaw including a first proximally projecting beam, the distally facing surface of the body of the knife assembly being configured to engage the first proximally projecting beam when body of the knife assembly is at the distal position.

Example 17

The apparatus of Example 16, the shaft assembly further including an inner guide, the inner guide including a laterally facing surface and a distally facing surface; the second jaw further including a second proximally projecting beam, the second proximally projecting beam being positioned and configured to engage the laterally facing surface of the inner guide to thereby establish and maintain a predetermined angular position of the end effector relative to the shaft assembly, the second proximally projecting beam being further positioned and configured to engage the distally facing surface of the inner guide to thereby establish and maintain a predetermined longitudinal position of the end effector relative to the shaft assembly.

Example 18

A method comprising: (a) positioning an end effector relative to a first fixture, the first fixture having a distally facing surface, the end effector having one or more ribs with a pair of proximally facing surfaces, the act of positioning the end effector comprising engaging the proximally facing surfaces of the end effector with the distally facing surface of the first fixture; (b) positioning a shaft assembly relative to the end effector while maintaining engagement between the proximally facing surfaces of the end effector with the distally facing surface of the first fixture; and (c) securing a distal portion of the shaft assembly to the end effector while maintaining engagement between the proximally facing surfaces of the end effector with the distally facing surface of the first fixture.

Example 19

The method of Example 18, further comprising: (a) positioning the shaft assembly relative to a second fixture, the second fixture having a proximally facing surface, the shaft assembly having a pair of distally oriented side projections with respective distally facing surfaces, the act of positioning the shaft assembly relative to the second fixture comprising engaging the distally facing surfaces of the shaft assembly with the proximally facing surface of the second fixture; (b) positioning a body relative to the shaft assembly while maintaining engagement between the distally facing surfaces of the shaft assembly with the proximally facing surface of the second fixture; and (c) securing a proximal portion of the shaft assembly to the body while maintaining engagement between the distally facing surfaces of the shaft assembly with the proximally facing surface of the second fixture.

Example 20

The method of Example 19, the act of positioning the shaft assembly relative to the second fixture being performed after the act of securing the distal portion of the shaft assembly to the end effector.

IV. Miscellaneous

Any of the versions of the instruments described herein may include various other features in addition to or in lieu of those described above. While the examples herein are described mainly in the context of electrosurgical instruments, various teachings herein may be readily applied to a variety of other types of devices. By way of example only, the various teachings herein may be readily applied to other types of electrosurgical instruments, tissue graspers, tissue retrieval pouch deploying instruments, surgical staplers, surgical clip appliers, ultrasonic surgical instruments, etc. The teachings herein may be readily applied to any of the instruments described in any of the references cited herein. Any of the devices herein may also include one or more of the various features disclosed in any of the various references that are incorporated by reference herein. The teachings herein may thus be readily combined with the teachings of any of the references cited herein in numerous ways. Other types of instruments into which the teachings herein may be incorporated will be apparent to those skilled in the art in view of the teachings herein.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. The disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure.

The terms “proximal” and “distal” are defined herein relative to a surgeon, robotic arm, or other operator or structure grasping a surgical instrument having a distal surgical end effector. The term “proximal” refers the position of an element closer to the surgeon, robotic arm, or other operator or structure; and the term “distal” refers to the position of an element closer to the surgical end effector of the surgical instrument and further away from the surgeon or other operator or structure.

As used herein, an element or step recited in the singular and proceeded with the word “a” or “an” should be understood as not excluding plural of said elements or steps, unless such exclusion is explicitly stated. Furthermore, references to “one example” or “an example” are not intended to be interpreted as excluding the existence of additional examples that also incorporate the recited features. The use of “including,” “comprising,” or “having” and variations thereof herein is meant to encompass the items listed thereafter, and equivalents thereof, as well as additional items.

As used herein in the specification and claims, including as used in the examples and unless otherwise expressly specified, the terms “about” or “approximately” for any numerical values or ranges indicate a suitable dimensional tolerance, or other form of reasonable expected range, that allows the part or collection of components to function for its intended purpose as described herein. More specifically, “about” or “approximately” may refer to the range of values that are within ±10% of the recited value (e.g., “about 100” may refer to the range of values from 90 to 110, including 90, 110, 100, and all other values within the range of 90 and 110). Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. The terms “approximately” and “about” are thus utilized herein to represent the degree by which a quantitative representation may vary from a stated reference without resulting in a change in the basic function of the subject matter at issue.

The term “substantially” is also utilized herein to represent the degree by which a quantitative representation may vary from a stated reference without resulting in a change in the basic function of the subject matter at issue. The term “substantially” shall therefore be understood to include a range of conditions or results that provide a functional equivalent to an explicitly stated condition or result. For instance, if a task is “substantially complete,” the result of the task having been substantially completed is functionally equivalent to the result that would have been achieved if the task had been perfectly completed. As another non-limiting example, a component that is “substantially straight” or “substantially flat,” an apparatus including a component that is “substantially straight” or “substantially flat” may provide a result or effect that is functionally equivalent to a result or effect that would be achieved by the same apparatus including the same component in a perfectly straight or perfectly flat configuration. The range implied by the term “substantially” should also be read to include the perfect result that is within that range. Thus, the term “substantially complete” shall be read as including “perfectly complete” while also including a range of completeness that is functionally equivalent to perfectly complete. As another example, terms such as “substantially straight” and “substantially flat” shall be read as including “perfectly straight” and “perfectly flat,” respectively; while also including a range of straightness or flatness that is functionally equivalent to perfectly straight or flat, respectively. As with the terms “approximately” and “about,” the term “substantially” may indicate a suitable dimensional tolerance, or other form of reasonable expected range, that allows a part or collection of components to function for its intended purpose as described herein.

The limitations of the following claims are not written in means-plus-function format and are not intended to be interpreted based on 35 U.S.C. §112(f), unless such claim limitations expressly use the phrase “means for” followed by a statement of function void of further structure.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

What is claimed is:

1. An apparatus comprising:
(a) body;
(b) a shaft assembly defining a longitudinal axis, the shaft assembly including an inner guide, the inner guide including a laterally facing surface and a distally facing surface; and
(c) an end effector, the end effector including:
(i) a first jaw, and
(ii) a second jaw, the first jaw being movable toward and away from the second jaw, the second jaw including a first proximally projecting beam,
the first proximally projecting beam being positioned and configured to engage the laterally facing surface of the inner guide to thereby establish and maintain a predetermined angular position of the end effector relative to the shaft assembly,
the first proximally projecting beam being further positioned and configured to engage the distally facing surface of the inner guide to thereby establish and maintain a predetermined longitudinal position of the end effector relative to the shaft assembly.

2. The apparatus of claim 1, the distally facing surface of the inner guide being positioned at a proximal end of the laterally facing surface.

3. The apparatus of claim 1, the shaft assembly further including an outer tube, the inner guide being disposed within the outer tube, first proximally projecting beam being radially interposed between the inner guide and an inner surface of the outer tube, the inner guide being radially interposed between the first proximally projecting beam and the longitudinal axis.

4. The apparatus of claim 1, the inner guide being laterally offset relative to the longitudinal axis.

5. The apparatus of claim 1, the shaft assembly further including a knife assembly having a knife, the knife being movable through one or both of the first jaw or the second jaw.

6. The apparatus of claim 5, the knife assembly further including a body extending through the shaft assembly, the body of the knife assembly being translatable relative to the inner guide between a proximal position and a distal position.

7. The apparatus of claim 6, the body of the knife assembly having a distally facing surface, the second jaw including a second proximally projecting beam, the distally facing surface of the body of the knife assembly being configured to engage the second proximally projecting beam when body of the knife assembly is at the distal position.

8. The apparatus of claim 7, the second jaw defining a knife slot having a distal end, the knife being translatable along the knife slot of the second jaw, the distally facing surface of the body of the knife and the second proximally projecting beam being configured and arranged to prevent the knife from reaching the distal end of the knife slot when the body of the knife assembly is in the distal position.

9. The apparatus of claim 7, the second proximally projecting beam being laterally positioned relative to the first proximally projecting beam on an opposite side of the longitudinal axis.

10. The apparatus of claim 7, the second proximally projecting beam being substantially parallel with the first proximally projecting beam.

11. The apparatus of claim 7, the first proximally projecting beam having a first length, the second proximally projecting beam having a second length, the second length being approximately equal to the first length.

12. The apparatus of claim 7, the shaft assembly further including an outer tube, the outer tube having a distal end with a first distally oriented side projection, and a second distally oriented side projection, the first distally oriented side projection being spaced apart from the second distally oriented side projection, the first distally oriented side projection being secured to the first proximally projecting beam, the second distally oriented side projection being secured to the second proximally projecting beam.

13. The apparatus of claim 12, the first distally oriented side projection having an exposed first distally facing surface, the second distally oriented side projection having an exposed second distally facing surface, the first and second distally facing surfaces being positioned and configured to engage a proximally facing surface of a fixture.

14. The apparatus of claim 1, the second jaw including an outwardly extending rib, the rib having an exposed proximally facing surface, the proximally facing surface being positioned and configured to engage a distally facing surface of a fixture.

15. The apparatus of claim 1, the end effector further including one or more electrodes operable to apply radiofrequency energy to tissue.

16. An apparatus comprising:
(a) body;
(b) a shaft assembly defining a longitudinal axis, the shaft assembly including; a knife assembly having a knife and a body, the knife being movable through one or both of the first jaw or the second jaw, the body of the knife assembly being translatable between a proximal position and a distal position, the body of the knife assembly having a distally facing surface; and
(c) an end effector, the end effector including:
(i) a first jaw, and
(ii) a second jaw, the first jaw being movable toward and away from the second jaw, the second jaw including a first proximally projecting beam,
the distally facing surface of the body of the knife assembly being configured to engage the first proximally projecting beam when body of the knife assembly is at the distal position.

17. The apparatus of claim 16, the shaft assembly further including an inner guide, the inner guide including a laterally facing surface and a distally facing surface;
the second jaw further including a second proximally projecting beam,
the second proximally projecting beam being positioned and configured to engage the laterally facing surface of the inner guide to thereby establish and maintain a predetermined angular position of the end effector relative to the shaft assembly,
the second proximally projecting beam being further positioned and configured to engage the distally facing surface of the inner guide to thereby establish and maintain a predetermined longitudinal position of the end effector relative to the shaft assembly.

18. A method comprising:
(a) positioning an end effector relative to a first fixture, the first fixture having a distally facing surface, the end effector having one or more ribs with a pair of proximally facing surfaces, the act of positioning the end effector comprising engaging the proximally facing surfaces of the end effector with the distally facing surface of the first fixture;
(b) positioning a shaft assembly relative to the end effector while maintaining engagement between the proximally facing surfaces of the end effector with the distally facing surface of the first fixture; and
(c) securing a distal portion of the shaft assembly to the end effector while maintaining engagement between the proximally facing surfaces of the end effector with the distally facing surface of the first fixture.

19. The method of claim 18, further comprising:
(a) positioning the shaft assembly relative to a second fixture, the second fixture having a proximally facing surface, the shaft assembly having a pair of distally oriented side projections with respective distally facing surfaces, the act of positioning the shaft assembly relative to the second fixture comprising engaging the distally facing surfaces of the shaft assembly with the proximally facing surface of the second fixture;
(b) positioning a body relative to the shaft assembly while maintaining engagement between the distally facing surfaces of the shaft assembly with the proximally facing surface of the second fixture; and
(c) securing a proximal portion of the shaft assembly to the body while maintaining engagement between the distally facing surfaces of the shaft assembly with the proximally facing surface of the second fixture.

20. The method of claim 19, the act of positioning the shaft assembly relative to the second fixture being performed after the act of securing the distal portion of the shaft assembly to the end effector.
